(19) United States
(12) Patent Application Publication
(10) Pub. No.: US 2025/0299783 A1
(43) Pub. Date: Sep. 25, 2025

(54) CALCULATION METHOD FOR PREDICTING GROUND STATE STRUCTURE OF SMALL T PEROVSKITE

(71) Applicants: SHANDONG AGRICULTURAL UNIVERSITY, Taian (CN); INSTITUTE OF PHYSICS, CHINESE ACADEMY OF SCIENCES, Beijing (CN)

(72) Inventors: Chao MA, Taian (CN); Guihua LI, Taian (CN); Kuijuan JIN, Beijing (CN); Kaimeng LIU, Taian (CN); Haodong SONG, Taian (CN); Yang LI, Taian (CN); Lin LI, Taian (CN); Yan WANG, Taian (CN); Lei GAO, Taian (CN); Xinghua LIU, Taian (CN)

(73) Assignees: SHANDONG AGRICULTURAL UNIVERSITY, Taian, SD (CN); INSTITUTE OF PHYSICS, CHINESE ACADEMY OF SCIENCES, Beijing (CN)

(21) Appl. No.: 18/826,228

(22) Filed: Sep. 6, 2024

(30) Foreign Application Priority Data
Mar. 21, 2024 (CN) 2024103274505

(51) Int. Cl. G16C 20/30; G16C 60/00

(52) U.S. Cl. CPC G16C 20/30; G16C 60/00

(57) ABSTRACT

A method for predicting the ground state structure of small τ perovskite is disclosed. The method includes: S1, determining the positional occupations of elements MI, MII, and O in the perovskite by constructing Pm3 m symmetric MIMIIO3 and MIIMIO3 structures. The total energies of the relaxed MIMIIO3 and MIIMIO3 structures are compared, and the structure with the lower energy is selected. S2, the ground state structure of the selected lower energy structure is determined using a branch prediction method. This method leverages the group-subgroup relationship of octahedral tilting, the minimum energy principle, and branch calculation strategies, simplifying the process by utilizing symmetry information and structural evolution of perovskite materials. The method requires only a few days, avoiding the inefficiencies of traditional approaches, which rely on generating numerous random structures. The invention improves the practicability, efficiency, and accuracy of predicting small τ perovskite ground state structures.

### TECHNICAL FIELD

The invention relates to the field of perovskite ground state structure prediction technology, in particular to a calculation method for predicting a ground state structure of small τ perovskite.

### BACKGROUND ART

Perovskite materials with small tolerance factors may have rare quantum ground states, such as the metallic ferroelectric state in LiOsO₃ (τ=0.861). Recent studies on small τ ferroelectric perovskite ZnSnO₃ (τ=0.814) have shown that the antiferroelectric distortion rotation modes of the oxygen octahedron can enhance the ferroelectric distortion. Therefore, the R3c structure can be stabilized with large ferroelectric polarization and highly tilted oxygen octahedrons, which indicates that it is possible to find more rare quantum ground states from small τ perovskite materials.

Traditional crystal structure prediction methods, such as the particle swarm evolution algorithm used in Calypso software, need to specify the maximum number of atoms of the lattice first when applied to predicting perovskite structure with a small tolerance factor of selected elements. For example, when the maximum number of atoms is limited to 10, Calypso software will generate more than 2000 crystal structures during the structural evolution process, and each structure needs to be completely relaxed and calculates the total energy, the calculation process is complex. If the lattice of the ground state structure exceeds 10 atoms, the calculation amount will be more complicated.

Due to the large anion radius, most perovskite nitrides, chalcogenides, and halides have small tolerance factors. However, their ground-state structures are very complex. Traditional crystal structure prediction methods need to generate a large number of random structures, which is very time-consuming. In order to determine the ground state structure of a given element type in ABX₃ compounds, a branch prediction method is developed.

### SUMMARY OF THE INVENTION

The purpose of this invention is to disclose a calculation method for predicting a ground state structure of small τ perovskite to determine the ground state structure of a given element type in small τ perovskite ABX₃-type compounds.

To achieve the above purpose, the present invention discloses a calculation method for predicting a ground state structure of small τ perovskite, comprising the following steps:
S1, determining position occupations of elements M_{I}, M_{II}, and O in perovskite:
constructing Pm3̅m symmetric M_{I}M_{II}O₃ and M_{II}M_{I}O₃ structures, and comparing total energies of M_{I}M_{II}O₃ and M_{II}M_{I}O₃ after a complete relaxation, and selecting the structure with a lower total energy;
S2, determining a ground state structure of the structure with the lower total energy in S1 by a branch prediction method.

Preferably, when a total energy of M_{I}M_{II}O₃ and M_{II}M_{I}O₃ after complete relaxation is close, finding quasi-ground state structures of M_{I}M_{II}O₃ and M_{II}M_{I}O₃ by the branch prediction method respectively, and comparing total energies of the quasi-ground state structure after complete relaxation, and selecting the structure with a lower total energy as a ground state structure.

The branch prediction method comprises:
S2-1, selecting six sub-group structures of Pm3̅m structure, the six sub-group structures comprise two main branch structures of MB_{R} and MB_{R+D};
the perovskite in the main branch MB_{R} contains certain rotation modes of BX₆ octahedron, and the main branch MB_{R} comprises two branches of B_{R} and B_{R+C}; B_{R} comprises a subgroup structure R3̅c, B_{R+C} comprises a subgroup structure Im3̅; compared with the B_{R} branch, the B_{R+C} branch structure also contains a change in the perovskite A-site coordination environment;
the perovskite in the main branch MB_{R+D} contains distortion modes of rotations, bond-length, and bond-angle of the BX₆ octahedron, the main branch MB_{R+D} comprises two branches of B_{R+D} and B_{R+D+C}; B_{R+D} comprises subgroup structures of Imma, P4/mbm and I4/mcm, B_{R+D+C} comprises a subgroup structure I4/mmm; compared with the B_{R+D} branch, the B_{R+D+C} branch structure also contains a change in the perovskite A-site coordination environment;
selecting a branch in B_{R}, B_{R+C}, B_{R+D}, and B_{R+D+C} for calculation; if a structure with a lowest energy in subgroup structures of the chosen branch is not dynamically stable, continue to select the structure with the lowest energy to calculate the structure with the lowest energy in the subgroup structures; if the structure with the lowest energy is still not dynamically stable, it needs to be solved until a dynamically stable structure with the lowest energy in the subgroup structure of the branch is found as a ground state structure of the branch.

S2-2, selecting a structure with the lowest energy in the ground state structure of B_{R}, B_{R+C}, B_{R+D}, and B_{R+D+C} as a ground state structure of perovskite.

Preferably, in S2-1, if a branch structure that contains a change in the perovskite A-site coordination environment does not have a significant energy reduction, it is unnecessary to calculate the branch.

Preferably, in S2-1 and S2-2, dynamic stability means that a phonon spectrum of the subgroup structure has no imaginary frequency.

The Pm3̅m structure has the highest symmetry in perovskite materials, although some perovskite materials may not change to the Pm3̅m phase before melting directly at high temperatures, the Pm3̅m structure can be constructed theoretically.

When the small r perovskite material gradually cools from the high temperature and high symmetry phase, some tilt patterns of the oxygen octahedron will be triggered. Glazer initially developed the Glazer symbol to represent different tilt patterns and derived a total of 23 patterns and their corresponding 15 space group structures. Howard and Stokes used group theory to analyze the group-subgroup relationship of these 15 spatial groups.

From the group-subgroup relationship of 15 space groups, it can be seen that when looking for the ground state structure, only 6 subgroups of the Pm3̅m structure need to be analyzed. Because if there are other tilt patterns or polar instability modes in the system, the corresponding imaginary frequency mode (soft mode phonon) will appear in the phonon spectrum, and the evolution direction of the structure can be determined according to the eigenvector of the soft mode phonon at high symmetric K-points in the reciprocal space.

Therefore, the invention adopts the above calculation method for predicting a ground state structure of small r perovskite, and it has the following technical effects:
(1) Combining the group-subgroup relationship of the octahedral tilt pattern in perovskite, the principle of minimum energy, and the branch calculation strategy, we make full use of the symmetry information and structural evolution characteristics of perovskite materials. Since only a few possible evolutionary branches are considered, it is usually only necessary to calculate about a dozen of (or dozens maximally) atomic structures to determine the ground state structure. It usually takes only a few days to obtain the ground state structure, which avoids the shortcomings of traditional methods that need to generate a large number of random structures and are time-consuming;
(2) It does not limit the number of atoms in the unit cell, so it has strong flexibility and can be used to search for systems with charge ordering and complex magnetic interactions;
(3) The branch prediction method in this invention realizes the practicability, efficiency, and accuracy of the ground state structure prediction of perovskite materials with small tolerance factors.

### DETAILED DESCRIPTION OF THE DRAWINGS

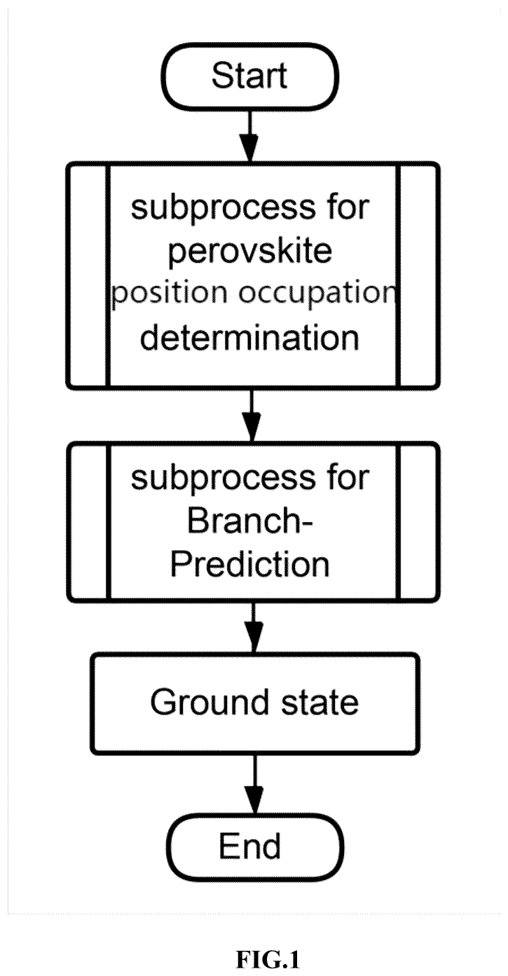

FIG. 1 is a brief calculation process of the calculation method for predicting the ground state structure of small τ perovskite;

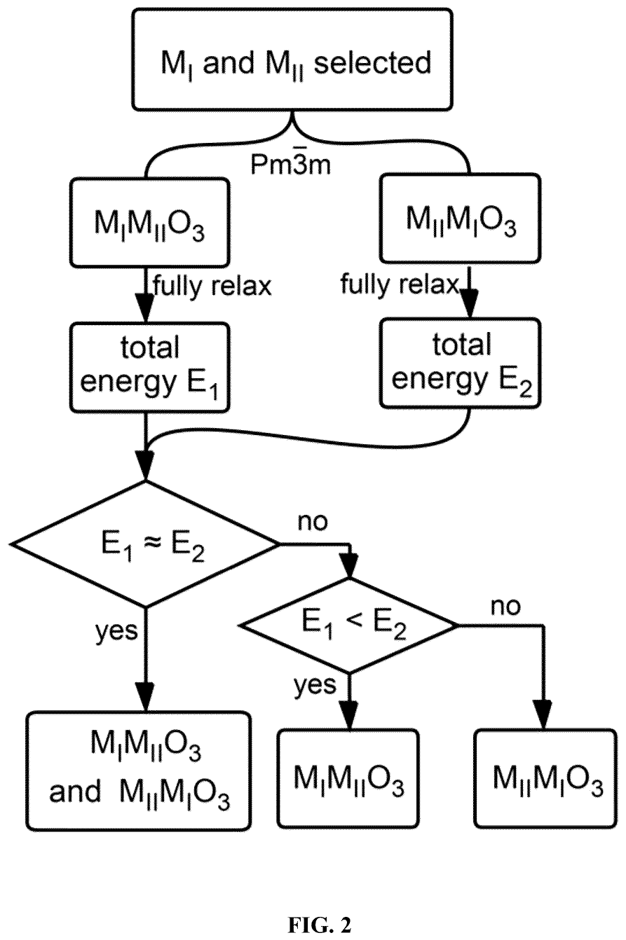

FIG. 2 is a calculation process for determining the position occupations of different elements in perovskite under given elements;

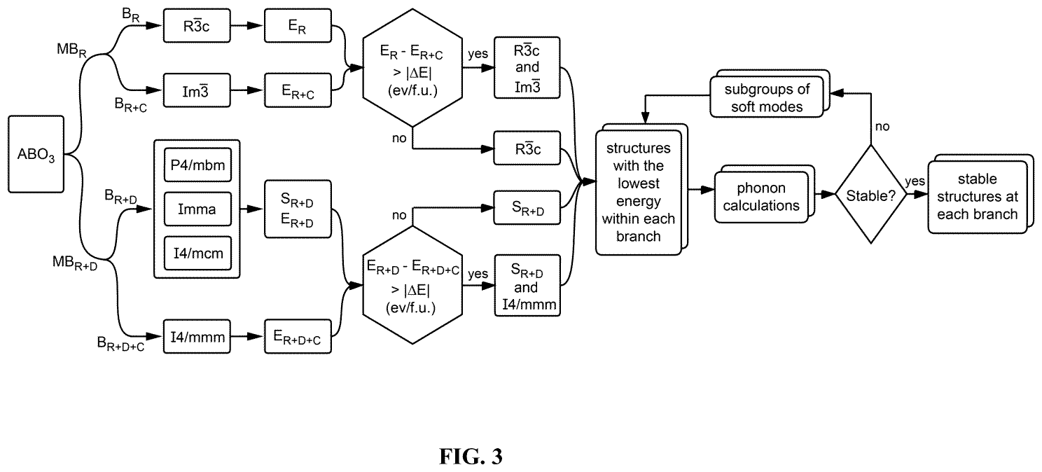

FIG. 3 is a detailed calculation process of the branch prediction sub-process;

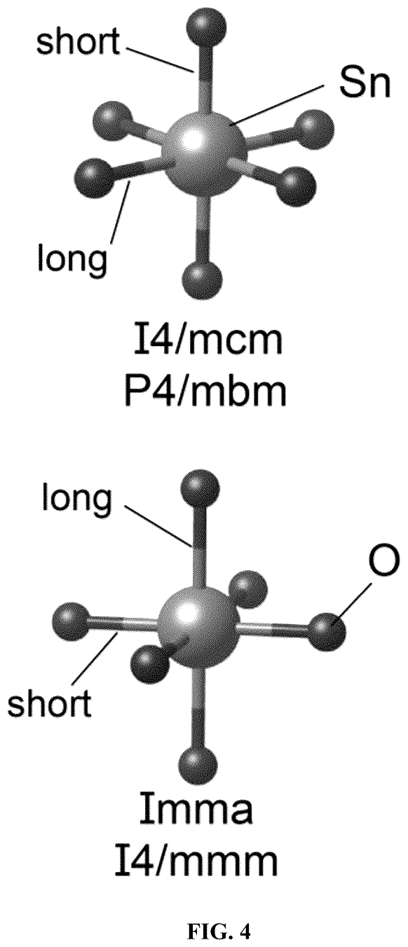

FIG. 4 is a distorted structure diagram of the bond-length in the four space group structures of the MB_{R+D} main branch;

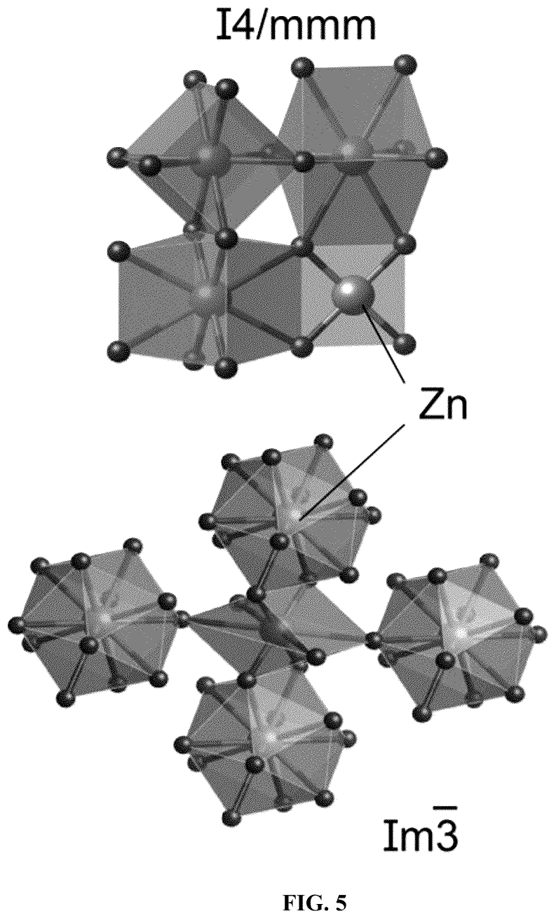

FIG. 5 is a schematic diagram of the change of the perovskite A-site coordination environment in the Im3̅ and I4/mmm structures;

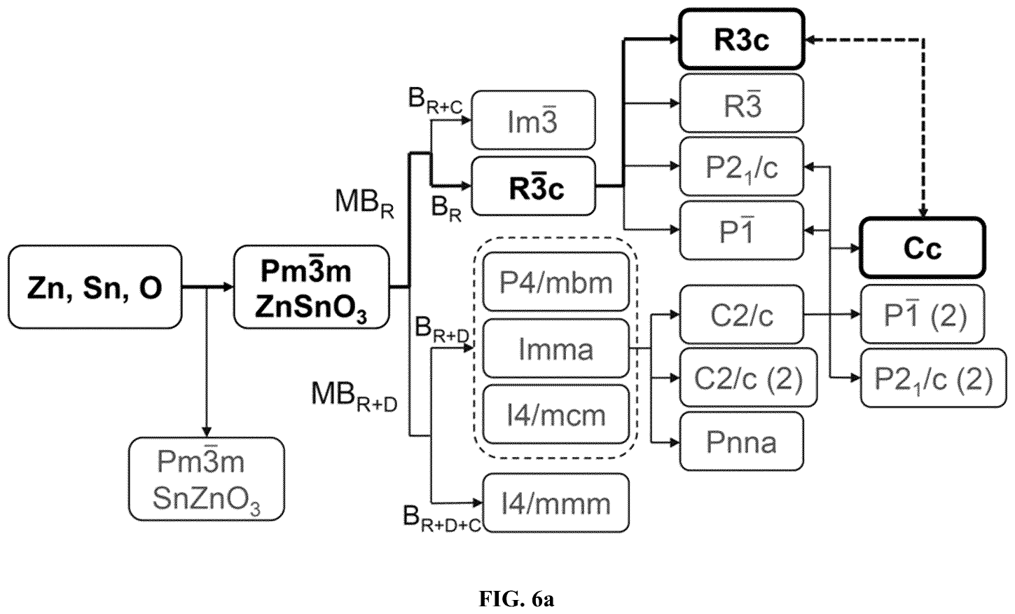

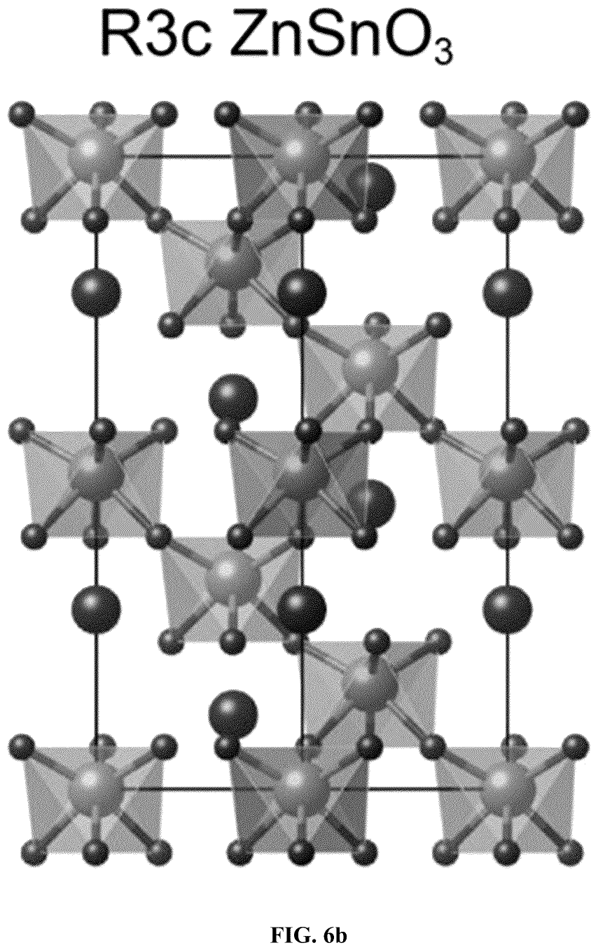

FIG. 6a is a calculation process of the ground state structure of the perovskite material system composed of (Zn, Sn, O);

FIG. 6b is a ground state structure diagram of the perovskite material system composed of (Zn, Sn, O);

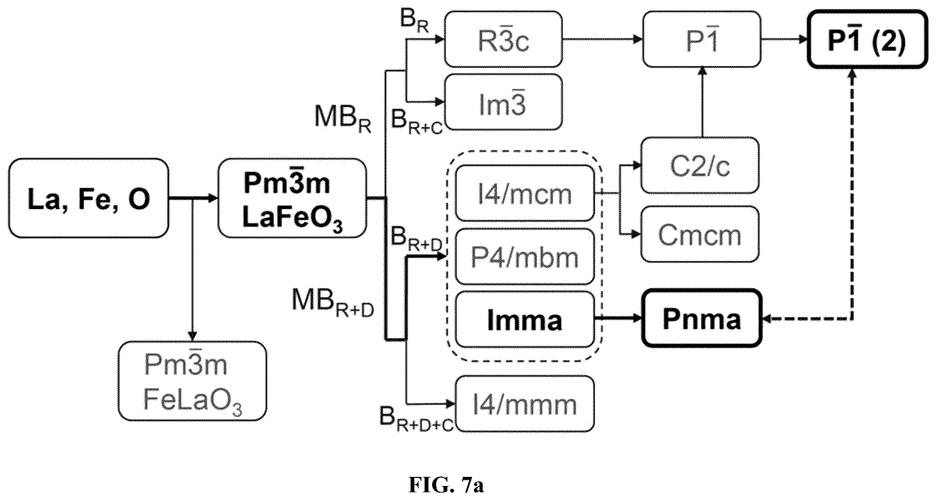

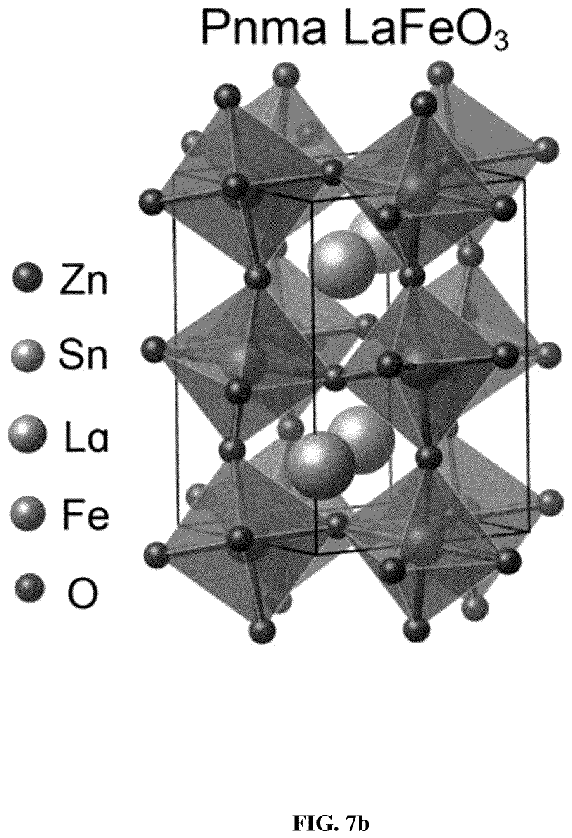

FIG. 7a is a calculation process of the ground state structure of the perovskite material system composed of (La, Fe, O);

FIG. 7b is a ground state structure diagram of the perovskite material system composed of (La, Fe, O);

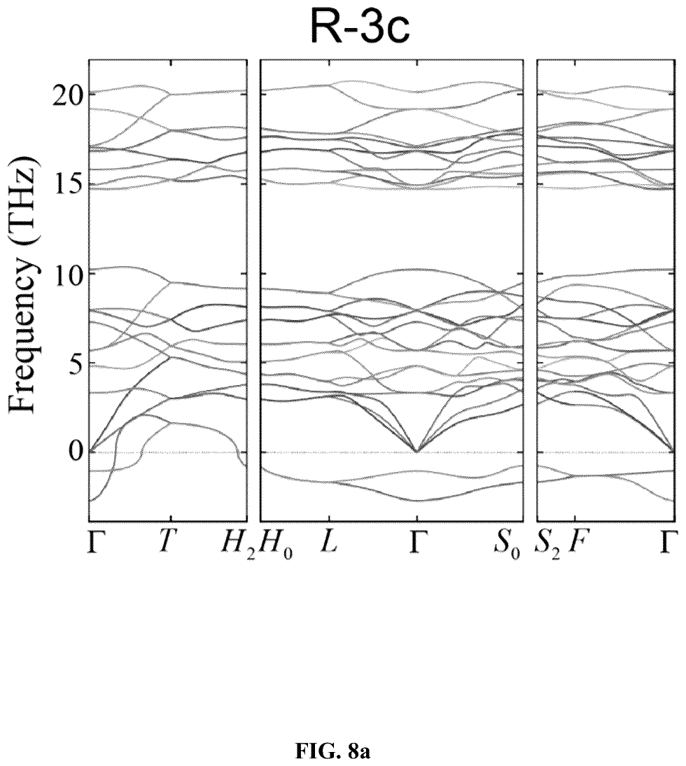

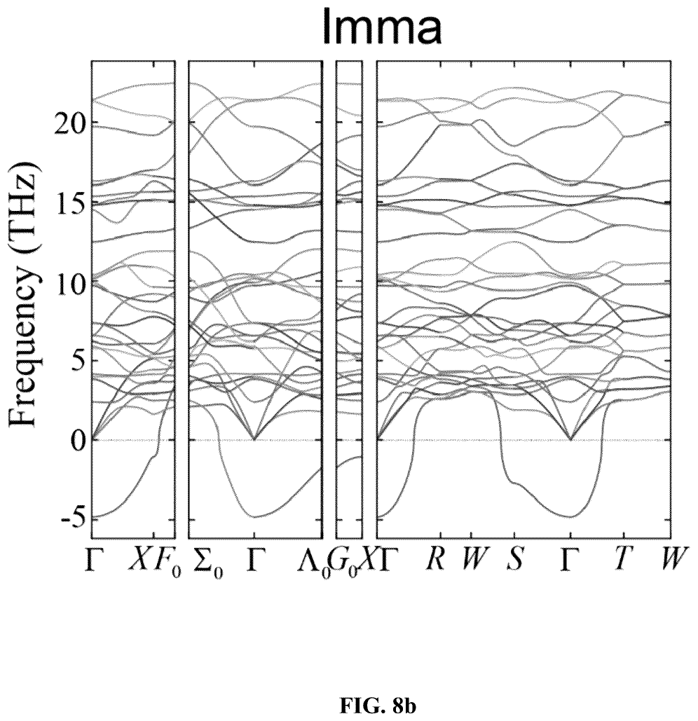

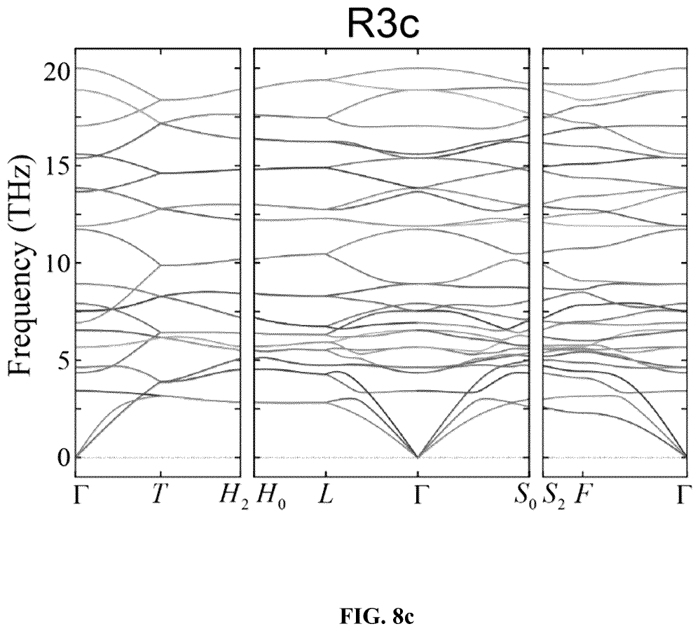

FIG. 8a is a phonon spectrum of R3̅c in the branch prediction process of the perovskite material system composed of (Zn, Sn, O);

FIG. 8b is a phonon spectrum of Imma in the branch prediction process of the perovskite material system composed of (Zn, Sn, O);

FIG. 8c is a phonon spectrum of R3c in the branch prediction process of the perovskite material system composed of (Zn, Sn, O);

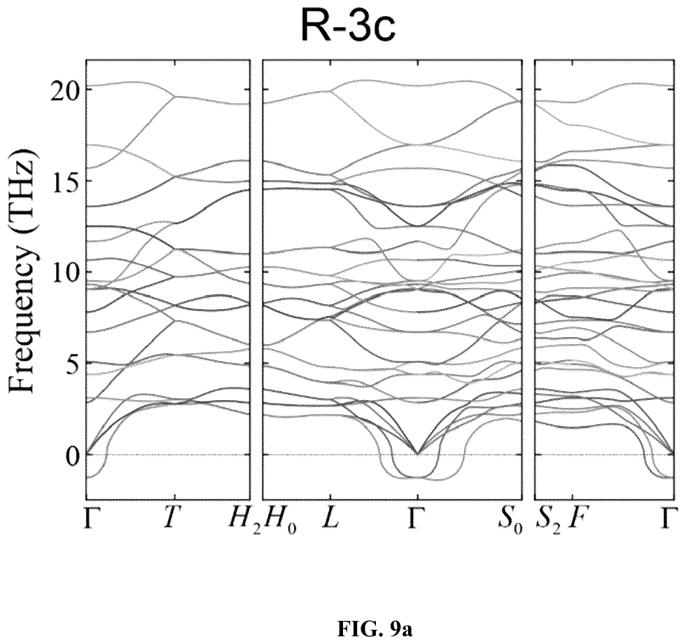

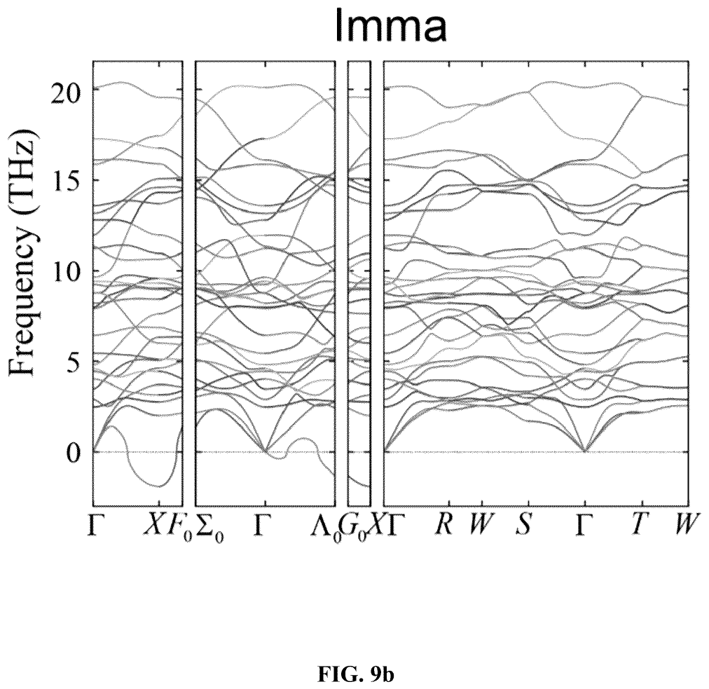

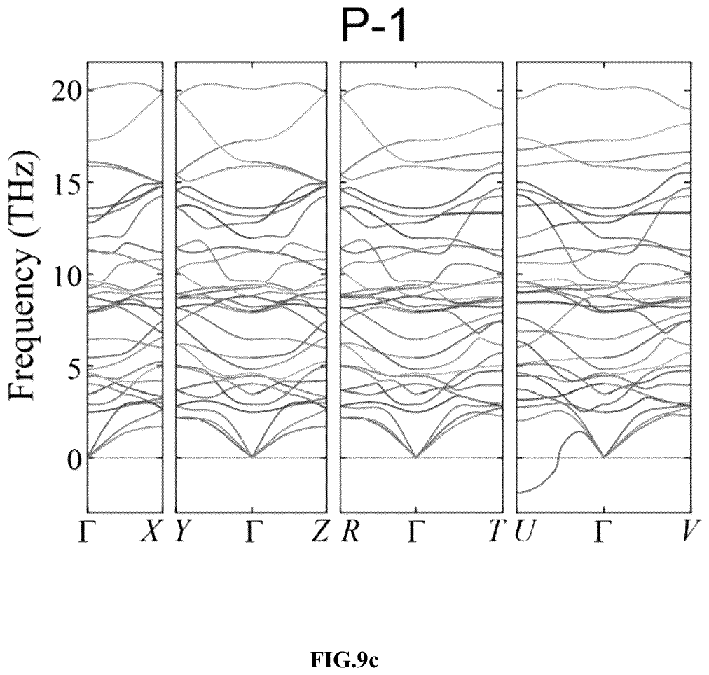

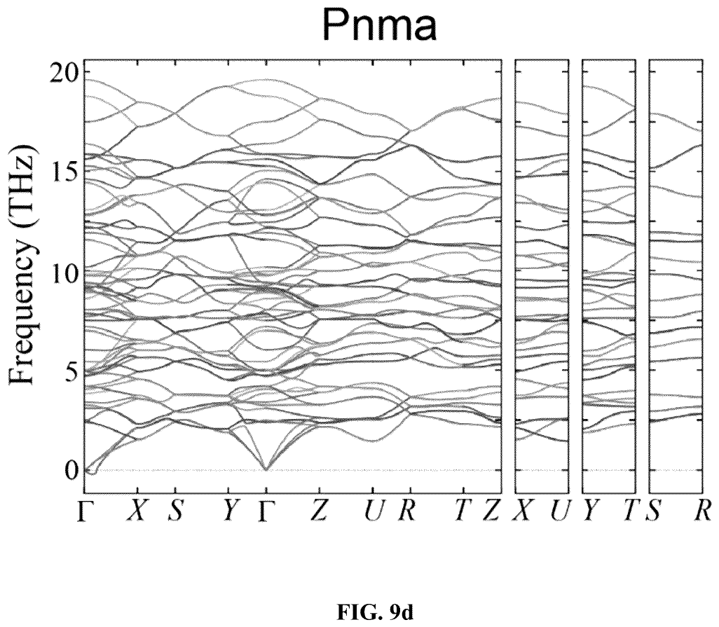

FIG. 9a is a phonon spectrum of R3̅c in the branch prediction process of the perovskite material system composed of (La, Fe, O);

FIG. 9b is a phonon spectrum of Imma in the branch prediction process of the perovskite material system composed of (La, Fe, O);

FIG. 9c is a phonon spectrum of P1̅ in the branch prediction process of the perovskite material system composed of (La, Fe, O);

FIG. 9d is a phonon spectrum of Pnma in the branch prediction process of the perovskite material system composed of (La, Fe, O).

### DETAILED DESCRIPTION OF THE INVENTION

The following is a further explanation of the technical scheme of the invention through drawings and embodiments.

Unless otherwise defined, the technical terms or scientific terms used in the invention should be understood by people with general skills in the field to which the invention belongs.

### Embodiment 1

A calculation method for predicting a ground state structure of small τ perovskite, taking perovskite oxide as an example, the calculation process is shown in FIG. 1.

In the case of given elements Zn, Sn, and O, it is first necessary to determine the position occupations of different elements in the perovskite. As shown in FIG. 2, the Pm3̅m symmetric ZnSnO₃ and SnZnO₃ structures are constructed, and the total energies of the two structures after a complete relaxation are compared.

The total energy after the complete relaxation of ZnSnO₃ is −14.126000 eV/f.u., and the total energy after the complete relaxation of SnZnO₃ is −13.347221 eV/f.u. It indicates that Zn preferentially occupies the A-site of perovskite and Sn occupies the B-site, so the SnZnO₃ system is directly excluded.

Then the branch prediction sub-process is used to search the ground state structure of ZnSnO₃, the detailed calculation process of the branch prediction sub-process is shown in FIG. 3, and the atomic structures of the six subgroups can be constructed by SpuDS software. These six subgroup structures of Pm3̅m can be divided into two main branches: rotation only and rotation+distortion, which are represented by MB_{R} and MB_{R+D}, respectively.

In the main branch of the rotation-only (MB_{R}), R3̅c and Im3̅ structures only contain the rotation of the SnO₆ oxygen octahedron, while the octahedron maintains the regular octahedron shape. In the four space group structures of the rotation+distortion (MB_{R+D}) main branch, the SnO₆ oxygen octahedron not only rotates but also is accompanied by the distortion of bond-length and bond-angle.

The distortion of bond-length is shown in FIG. 4. There are two short Sn—O bonds and four long Sn—O bonds in the I4/mcm and P4/mbm SnO₆ octahedra, and two long Sn—O bonds and four short Sn—O bonds in Imma and I4/mmm SnO₆ oxygen octahedrons.

As shown in FIG. 5, there is also a change in the perovskite A-site coordination environment in the Im3̅ and I4/mmm structure, so the main branch of rotation only (MB_{R}) can also be divided into two branches of B_{R}(R3̅c) and B_{R+C}(Im3̅), and at the same time, the main branch of rotation+distortion (MB_{R+D}) can be divided into two branches of B_{R+D} (Imma, P4/mbm and I4/mcm) and B_{R+D+C} (I4/mmm). So far, the structures contained in each branch have specific similar features.

In general, all subspace groups under two branches of the same main branch need to be calculated. However, if the branch of the A-site coordination environment does not bring a significant energy reduction, it shows that this change does not have an energy advantage. Therefore, the branch doesn't need further calculations, and the calculation process can be simplified to a certain extent.

The B_{R+D} branch contains three structures: Imma, P4/mbm, and I4/mcm. One or more structures need to be selected according to their energy as the representative of the R+D structure to continue the calculation. The structure is expressed as S_{R+D} in FIG. 3.

If there is a structure with the lowest energy and dynamic stability (no imaginary frequency in the phonon spectrum) in all subspace groups of all branches, then this structure is the ground state structure of the system. If the lowest energy structure is unstable (imaginary frequency exists in the phonon spectrum), then its subspace group needs to be calculated until the ground state structure is searched.

The branch prediction process applied to (Zn, Sn, O) is shown in part a of FIG. 6. The group-subgroup relationship of the ZnSnO₃ branch prediction process and the corresponding energy of each space group structure are shown in Table 1:

TABLE 1
The group-subgroup relationship of the branch prediction process of
ZnSnO₃ and the energy corresponding to the structure of each space group

| | Energy | | Space | Energy | |
|---|---|---|---|---|---|
| ABO₃ | (eV/f.u.) | Main branch | group | (eV/f.u.) | Stable? |
| Pm-3m | −14.126000 | Rotation only | Im3̅ | −17.069102 | |
| ZnSnO₃ | | | R3̅c | −17.819789 | no |
| | | Rotation + | I4/mmm | −16.872062 | |
| | | distortion | P4/mbm | −15.986270 | |
| | | | Imma | −17.521654 | no |
| | | | 14/mcm | −16.017827 | |

| Subgroup level1 | | | Subgroup level2 | | |
|---|---|---|---|---|---|
| Space | Energy | | Space | Energy | |
| group | (eV/f.u.) | Stable? | Group | (eV/f.u.) | Stable? |
| R3c | −17.967395 | yes | | | |
| R-3 | −17.839290 | | | | |
| P-1 (1) | −17.878869 | | | | |
| P2₁/c (1) | −17.882555 | | | | |
| C2/c | −17.819787 | no | Cc | To R3c | yes |
| | | | P-1 (2) | −17.839230 | |
| | | | P2₁/c (2) | −17.843207 | |
| | | | P2₁/c (1) | −17.882555 | |
| | | | P-1 (1) | −17.878869 | |
| C2/c (2) | −17.618737 | | | | |
| Pnna | −17.523913 | | | | |

The ground state structure of ZnSnO₃ (τ=0.814) is predicted to be an R3c structure, and the corresponding atomic structure is shown in FIG. 2. The phonon spectrum of the key intermediate structure in the prediction process is shown in FIG. 8. In FIG. 8, a is the phonon spectrum of R3̅c, b is the phonon spectrum of Imma, and c is the phonon spectrum of R3c.

### Embodiment 2

The ground state structure of the perovskite material system composed of (La, Fe, O) is determined by the same method as the Embodiment.

The total energy of FeLaO₃ after complete relaxation is −21.743328 eV/f.u., and the total energy of LaFeO₃ after complete relaxation is −30.398413 eV/fu., indicating that La preferentially occupies the A site of perovskite and Fe occupies the B site, so the FeLaO₃ system is directly excluded.

The branch prediction method for LaFeO₃ is shown in part a in FIG. 7. The ground state structure of LaFeO₃ (τ=0.954) is predicted to be a Pnma structure, and the corresponding atomic structure is shown in part b in FIG. 7.

The phonon spectrum of the key intermediate structure in the prediction process is shown in FIG. 9. Part a in FIG. 9 is a phonon spectrum of R3̅c; part b in FIG. 9 is a phonon spectrum of Imma; part c in FIG. 9 is a phonon spectrum of P1̅; part d in FIG. 9 is a phonon spectrum of Pnma.

The energy of the subgroup structure in the branch prediction method flow of LaFeO₃ expansion is shown in Table 2:

TABLE 2
The group-subgroup relationship of the LaFeO₃ branching prediction process
and the corresponding energy of each spatial group structure

| | Energy | | Space | Energy | |
|---|---|---|---|---|---|
| ABO₃ | (eV/f.u.) | Main branch | group | (eV/f.u.) | Stable? |
| Pm-3m | −30.398413 | Rotation only | Im3̅ | −30.615945 | |
| LaFeO₃ | | | R3̅c | −31.032246 | no |
| | | Rotation + | 14/mmm | −30.820812 | |
| | | distortion | P4/mbm | −30.819776 | |
| | | | Imma | −31.040602 | no |
| | | | 14/mcm | −31.004435 | no |

| Subgroup level 1 | | | Subgroup level 2 | | |
|---|---|---|---|---|---|
| Space | Energy | | Space | Energy | |
| group | (eV/f.u.) | Stable? | group | (eV/f.u.) | Stable? |
| P-1 | −31.040628 | no | P-1 (2) | To Pnma | yes |
| Pnma | −31.065007 | yes | | | |
| C2/c | −31.032636 | no | P-1 | −31.040628 | |
| Cmcm | −31.012516 | | | | |

### Analysis

(1) As shown in Table 3, the lattice structures and atomic positions calculated by Embodiment 1 and Embodiment 2 are consistent with those in Reference (1) Inaguma, Y, Yoshida, M. & Katsumata, T. A Polar Oxide ZnSn₃ with a LiNbO₃-Type Structure. J. Am. Chem. Soc. 130, 6704-6705 (2008) and Reference (2) Dixon, C. A. L. et al. Thermal evolution of the crystal structure of the orthorhombic perovskite LaFeO₃. J. Solid State Chem. 230, 337-342 (2015), indicating that the branch prediction method is effective and reliable in searching for the ground state structure of small τ perovskite-related materials.

TABLE 3
Comparison of calculated and experimental structural parameters
of hexagonal-R3c (H-R3c) ZnSnO₃ and orthorhombic-Pnma LaFeO₃

| Material | Crystal lattice | Atom | WP | x | y | z | Reference |
|---|---|---|---|---|---|---|---|
| ZnSnO₃ | H-R3c (No. 161) | Zn | 6a | 0 | 0 | 0.28467 | Embodiment |
| (Calculation) | a = 5.25116 Å | Sn | 6a | 0 | 0 | 0 | 1 |
| | c = 14.00065 Å | O | 18b | 0.032 | 0.36219 | 0.06691 | |
| ZnSnO₃ | H-R3c (No. 161) | Zn | 6a | 0 | 0 | 0.2859 | (1) |
| (Experiment) | a = 5.2622 Å | Sn | 6a | 0 | 0 | 0 | |
| | c = 14.0026 Å | O | 18b | 0.0405 | 0.35 | 0.0709 | |
| LaFeO₃ | Pnma (No. 62) | La | 4c | 0.03321 | 0.25 | −0.00745 | Embodiment |
| (Calculation) | a = 5.53934 Å | Fe | 4b | 0 | 0 | 0.5 | 2 |
| | b = 7.79733 Å | O1 | 4c | 0.48442 | 0.25 | 0.07646 | |
| | c = 5.51701 Å | O2 | 8d | 0.28387 | 0.04105 | 0.71687 | |
| LaFeO₃ | Pnma (No. 62) | La | 4c | 0.03128 | 0.25 | 0.0075 | (2) |
| (Temperature | a = 5.56272 Å | Fe | 4b | 0 | 0 | 0.5 | |
| 25 K | b = 7.84142 Å | O1 | 4c | 0.48509 | 0.25 | 0.0739 | |
| experiment) | c = 5.54631 Å | O2 | 8d | 0.28389 | 0.04006 | 0.71708 | |

Note:
WP is an abbreviation of Wyckoff's position.

(2) It can be seen from Embodiment 1 and Embodiment 2 that the ground state structure can be determined by finding the lowest energy and dynamically stable structure in the same subgroup level of different branches, without finding the dynamically stable structure of all different branches. Taking ZnSnO₃ as an example, when the R3c structure is found, it is not necessary to continue the calculation, so the subgroup of the C2/c structure does not need to be calculated, which greatly reduces the calculation process of the branch prediction method.

Therefore, the invention adopts the above-mentioned calculation method for predicting the ground state structure of small τ perovskite, combines the group-subgroup relationship of octahedral tilt pattern in perovskite, the principle of minimum energy and the branch calculation strategy, makes full use of the symmetry information and structural evolution characteristics of perovskite materials, simplifies the calculation process, and usually only needs a few days to determine the ground state structure, which avoids the shortcomings of traditional methods that need to generate a large number of random structures and time-consuming; the practicability, efficiency, and accuracy of the ground state structure prediction of small τ perovskite materials are realized.

Finally, it should be explained that the above embodiments are only used to explain the technical scheme of the invention rather than restrict it. Although the invention is described in detail concerning the better embodiment, the ordinary technical personnel in this field should understand that they can still modify or replace the technical scheme of the invention, and these modifications or equivalent substitutions cannot make the modified technical scheme out of the spirit and scope of the technical scheme of the invention.

1. A calculation method for predicting a ground state structure of small τ perovskite, comprising the following steps:
determining types of elements M_{I}, M_{II} in ABX₃ compounds contained in the small τ perovskite;
S1, determining position occupations of the elements M_{I}, M_{II}, and O in perovskite:
constructing Pm3̅m symmetric M_{I}M_{II}O₃ and M_{II}M_{I}O₃ structures, and comparing total energies of M_{I}M_{II}O₃ and M_{II}M_{I}O₃ after a complete relaxation, and selecting the structure with a lower total energy;
S2, determining a ground state structure of the structure with the lower total energy in S1 by a branch prediction method;
the branch prediction method comprises:
S2-1, selecting six sub-group structures of 15 spatial groups with a lower total energy structure, the six sub-group structures comprise two main branch structures of MB_{R} and MB_{R+D};
a perovskite B-site in the main branch structure of MB_{R} contains certain rotation modes of oxygen octahedron, and the main branch structure of MB_{R} comprises two branches of B_{R} and B_{R+C}; B_{R} comprises a subgroup structure R3c, B_{R+C} comprises a subgroup structure Im3̅; compared with the B_{R} branch, the B_{R+C} branch structure also contains a change in a perovskite A-site coordination environment;
the perovskite B-site in the main branch structure of MB_{R+D} contains distortion modes of rotations, bond-length, and bond-angle of the oxygen octahedron, the main branch structure of MB_{R+D} comprises two branches: B_{R+D} and B_{R+D+C}; B_{R+D} comprises subgroup structures of Imma, P4/mbm and I4/mcm, B_{R+D+C} comprises a subgroup structure I4/mmm; compared with the B_{R+D} branch, the B_{R+D+C} branch structure also contains the change in the perovskite A-site coordination environment;
selecting a branch in B_{R}, B_{R+C}, B_{R+D}, and B_{R+D+C} for calculation; if a structure with a lowest energy in subgroup structures of the chosen branch is not dynamically stable, continue to select the structure with the lowest energy to calculate a structure with the lowest energy in the subgroup structures; if the structure with the lowest energy is still not dynamically stable, solving until a dynamically stable structure with the lowest energy in the subgroup structure of the branch is found as a ground state structure of the branch;
S2-2, selecting a structure with the lowest energy in the ground state structure of B_{R}, B_{R+C}, B_{R+D}, and B_{R+D+C} as a ground state structure of perovskite.

2. The calculation method for predicting the ground state structure of small τ perovskite according to claim 1, wherein when a total energy difference of M_{I}M_{II}O₃ and M_{II}M_{I}O₃ after complete relaxation is lower than 5%, finding quasi-ground state structures of M_{I}M_{II}O₃ and M_{II}M_{I}O₃ by the branch prediction method respectively, and comparing total energies of the quasi-ground state structure after complete relaxation, and selecting the structure with a lower total energy as a ground state structure.

3. The calculation method for predicting the ground state structure of small τ perovskite according to claim 1, wherein in S2-1, if an energy reduction of a branch structure of the perovskite A-site coordination environment is lower than 5%, it is not necessary to calculate the branch.

4. The calculation method for predicting the ground state structure of small τ perovskite according to claim 1, wherein in S2-1 and S2-2, dynamic stability means that a phonon spectrum of the subgroup structure has no imaginary frequency.
